(19) United States
(12) Patent Application Publication
(10) Pub. No.: US 2019/0298901 A1
(43) Pub. Date: Oct. 3, 2019

(54) HETEROCHRONIC BLOOD EXCHANGE AS A MODALITY TO INFLUENCE MYOGENESIS, NEUROGENESIS, AND LIVER REGENERATION

(71) Applicant: THE REGENTS OF THE UNIVERSITY OF CALIFORNIA, Oakland, CA (US)

(72) Inventors: Irina M. Conboy, El Sobrante, CA (US); Michael J. Conboy, El Sobrante, CA (US); Ranveer Gathwala, San Ramon, CA (US); Melod Mehdipour, Mountain View, CA (US); Yan Liu, Berkeley, CA (US)

(73) Assignee: THE REGENTS OF THE UNIVERSITY OF CALIFORNIA, Oakland, CA (US)

(21) Appl. No.: 16/381,878

(22) Filed: Apr. 11, 2019

Related U.S. Application Data
(63) Continuation of application No. PCT/US2017/056672, filed on Oct. 13, 2017.
(60) Provisional application No. 62/408,511, filed on Oct. 14, 2016.

(51) Int. Cl. A61M 1/02; A61M 1/34; A61B 5/02

(52) U.S. Cl. CPC A61M 1/024; A61M 1/3496; A61B 5/02042; A61M 2205/50; A61B 2503/42; A61M 2205/3379; A61M 2250/00; A61B 2503/40; A61M 2205/52

(57) ABSTRACT

A blood exchange system is provided that permits computer controlled isochronic and heterochronic blood exchange transfers for small animal model studies. The small animal blood exchange apparatus is an in vivo research tool to replace heterochronic parabiosis, a procedure that requires the suturing of skins of young and old mice (or animals of different genetic background, etc.) and the formation of shared circulatory systems. Compared to parabiosis, the in vivo animal study apparatus is faster, better controlled and is more flexible in the range of available and potential assays that can be performed. Blood exchange in small animals enables less invasive and better-controlled studies with more immediate translation to therapies for humans.

### CROSS-REFERENCE TO RELATED APPLICATIONS

This application claims priority to, and is a 35 U.S.C. § 111(a) continuation of, PCT international application number PCT/US2017/056672 filed on Oct. 13, 2017, incorporated herein by reference in its entirety, which claims priority to, and the benefit of, U.S. provisional patent application Ser. No. 62/408,511 filed on Oct. 14, 2016, incorporated herein by reference in its entirety. Priority is claimed to each of the foregoing applications.

The above-referenced PCT international application was published as PCT International Publication No. WO 2018/071869 A1 on Apr. 19, 2018, which publication is incorporated herein by reference in its entirety.

### STATEMENT REGARDING FEDERALLY SPONSORED RESEARCH OR DEVELOPMENT

This invention was made with Government support under Grant No. AG048316, awarded by the National Institutes of Health. The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to diagnostic or research devices methods, and more particularly an apparatus and methods for heterochronic blood exchange and isochronic blood exchange.

### 2. Background Discussion

The medical costs of managing an aging world poses significant economic and social challenges and will ultimately require a long-term solution. One reason for waning capabilities in people with advancing age is a progressive decline in organ function. One way to increase healthy longevity would be to rejuvenate the regenerative and repair capacity of aged tissues. Previous work suggests restoring the circulatory environment of aged tissues back to a productive, young composition may help to rapidly and broadly enhance the maintenance and repair of multiple old organs, combat degeneration and extend health span.

Heterochronic parabiosis is an experimental model where the vasculature of two animals of different ages are surgically joined together to create a shared circulatory system and has been used in stem cell and aging research in the last few decades. Heterochronic parabiosis has been shown to rejuvenate the performance of stem cells from old tissues at some expense to the young subject, but whether this occurs as a result of shared circulatory factors or shared organ systems is unclear.

The effects seen by heterochronic parabiosis include rejuvenation of multiple tissues in the old partner, and are often simplistically assumed to be caused by the exchange of macromolecules found in plasma; however, parabiosis is far more complex. For example, old animals with young partners have, through the shared circulation, continuous access to the young organs, which regulate metabolic homeostasis, wound clearance and inflammation, and provide blood oxygenation to the animals. Old mice attached to young animals also benefit from environmental enrichment and youthful pheromones, which may play a role in neuronal plasticity and neurogenesis. The young parabiont partially maintains an additional aged body with deteriorating organs, chronic inflammation, and skewed immune responses. Additionally, young and old organ systems have an opportunity to “adapt” to prolonged sharing of circulatory milieus and thus change their local influences on resident stem cells. All of the above could contribute to the observed differences in regenerative responses.

One conclusion from recent studies on heterochronic parabiosis is that the regenerative capacity of old tissue stem cells in all three germ layer derivatives can be enhanced by the young systemic milieu. It is tempting to assume that young plasma has pro-regenerative factors, and indeed administration of young plasma to aged mice improved their cognition. However, the effects of young blood plasma on stem cells in brain or other tissues have not been studied, and it remains to be discovered whether and which plasma factors would be active enough to influence neurogenesis or cognition at small doses when added to an aged circulation, and would be able to cross the blood brain barrier to have positive or negative central effects. Thus far, only heterochronic parabiosis has been shown to enhance myogenesis, hepatogenesis, bone regrowth, neurogenesis, cognition and the numbers of dendritic spines in old mice. Most importantly, the positive effects of heterochronic parabiosis are robust for muscle, lesser for liver and marginal for neurogenesis; and a significant inhibition of even young tissue stem cells by the aged circulatory milieu takes place.

Obviously the surgical joining of human circulatory systems is not a clinically adaptable approach. Accordingly, there is a need for a test bed that does not have the deficiencies and uncertainties of parabiosis where organs are shared as well as blood.

### BRIEF SUMMARY

The present technology provides an apparatus and method for isochronic and heterochronic blood and/or blood component exchanges in small animals. Heterochronic parabiosis has been shown to rejuvenate the performance of old tissue stem cells at some expense to the young, but whether this is through shared circulation or shared organs is unclear and parabiosis is not a clinically adaptable approach. The old heterochronic partners have access to young organs, environmental enrichment and youthful hormones/pheromones, while the young parabiont maintains an additional aged body with deteriorating organs.

To distinguish between the two, the present technology exchanges blood or blood components between young and old mice without sharing other organs and the other deficiencies of parabiosis. In contrast to parabiosis, where joint circulation is established in ˜7-10 days through growth of skin capillaries, blood exchange is instantaneous and well controlled by the device. The procedure is less invasive than parabiosis as it involves minimal surgery, only the catheterization of a jugular vein.

The apparatus provides a small animal blood exchange device and controlled experimental system where only blood or components are exchanged and the effects of potential therapeutic substances can be tested. The apparatus has a computer controlled microfluidic peristaltic pump circuit and computer and controlled extracorporeal blood manipulation system. The apparatus is the first to allow for continuous blood flow as required for larger scale experimental applications in live small animals.

The system has a pumping assembly with a computer control system and transfer tubes. In one embodiment, the pump assembly may also have a cell removal module that can remove red blood cells or other components that are returned to the donor to observe the separate humeral and cellular influences of heterochronic blood exchange. The computer may also have programming that controls the anesthesia system as well as the blood transfer conditions. Computer control over blood transfers avoids the risk of volume alterations and haemodynamic distress in both of the test subjects.

The apparatus and system also provide the opportunity for development and use of testing methods that comprehensively delineate the onset and duration of the positive and negative influences of the heterochronic system as well as identify blood factor(s) that influence health and uncover their mechanism of action.

For example, methods using the apparatus for identifying materials or processes that can lead to treatments that mitigate or reverse cellular and immune senescence or attenuate liver fibrosis and adiposity, muscle wasting and neuro-degeneration in old subjects.

Many methods for using the apparatus use the steps of (a) removing a volume of blood from a young individual; (b) removing a volume of blood from an old individual; and (c) replacing the removed volume of blood from the old individual with the volume of blood from the young individual; (d) wherein rapid liver regeneration, reduction of fibrosis, and muscle healing post-injury or immobility, or old age are induced. The heterochronic blood exchange may also be repeated at regular intervals over time before analysis.

Other methods for evaluating treatments and prevention of age-related diseases using the apparatus use the steps of: (a) removing a volume of blood from an old individual; (b) performing blood apheresis to remove deficient and deleterious blood components from the volume of old blood; (c) returning treated old blood to the old individual; and (d) performing a heterochronic blood exchange between the old individual and a young individual and studying the effects. The blood apheresis can also be repeated at regular intervals over time before analysis.

The effects of heterochronic blood exchange were examined in tissues from muscle, liver and brain hippocampus, in the presence and absence of muscle injury to illustrate the functionality of the system and methods. The effects were examined with respect to all three germ layer derivatives: injured-regenerating muscle, ongoing liver cell proliferation and brain-hippocampal neurogenesis, and in the presence and absence of muscle injury.

Surprisingly, the onset of the influence of heterochronic blood exchange on myogenesis, neurogenesis and hepatogenesis turned out to be amazingly fast, within a few days. In addition, the outcome of heterochronic blood exchange is also different from heterochronic parabiosis, particularly for neurogenesis where the results suggest that removal of “old blood components” is far more effective than adding young blood or blood components to the aged animal, and that peripheral tissue injury compounds the negative effects of old blood on young neurogenesis.

Blood exchange was shown to enhance old muscle repair without inhibition of the young, and old hepatogenesis is improved and fibrosis and adiposity are decreased, while young hepatogenesis becomes diminished. Moreover, the examples demonstrate a rapid increase in beta-2 microglobulin (B2M) in young tissues by old blood; and this phenotype is not from age-elevated circulating B2M (as there is none), suggesting that another age-specific systemic molecule raises B2M in the young organs.

Accordingly, the apparatus will allow the production of a variety of methods of treatment of humans that can ameliorate inhibitory factors present in aged blood and benefit from stimulatory factors carried by young blood.

According to one aspect of the technology, a blood exchange system is provided that permits computer controlled isochronic and heterochronic blood exchange transfers for small animal model studies.

Another aspect of the technology is to provide a system and methods for developing models and techniques for immediate translation to therapies for human such as continuous blood-exchange by blood-weight-volume ratio in clinical patients to induce rapid liver regeneration, reduction of fibrosis, muscle healing post-injury or immobility, or old age.

A further aspect is to provide a testing system for identifying positive factors and inhibitory blood components that can be removed to enhance neurogenesis, brain health, muscle regeneration, hepatogenesis and liver health, as well as physical performance and cognitive ability.

Another aspect of the technology is to provide an analytical system that can precisely determine the duration of the effects and optimal intervals between the treatments.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic illustration of and apparatus and system for small animal heterochronic or isochronic blood exchange according to one embodiment of the technology.

FIG. 2 is a functional block diagram of a method for heterochronic or isochronic blood exchange according to one embodiment of the technology.

FIG. 3 is a functional block diagram of an alternative method for heterochronic or isochronic blood exchange according to one embodiment of the technology.

### DETAILED DESCRIPTION

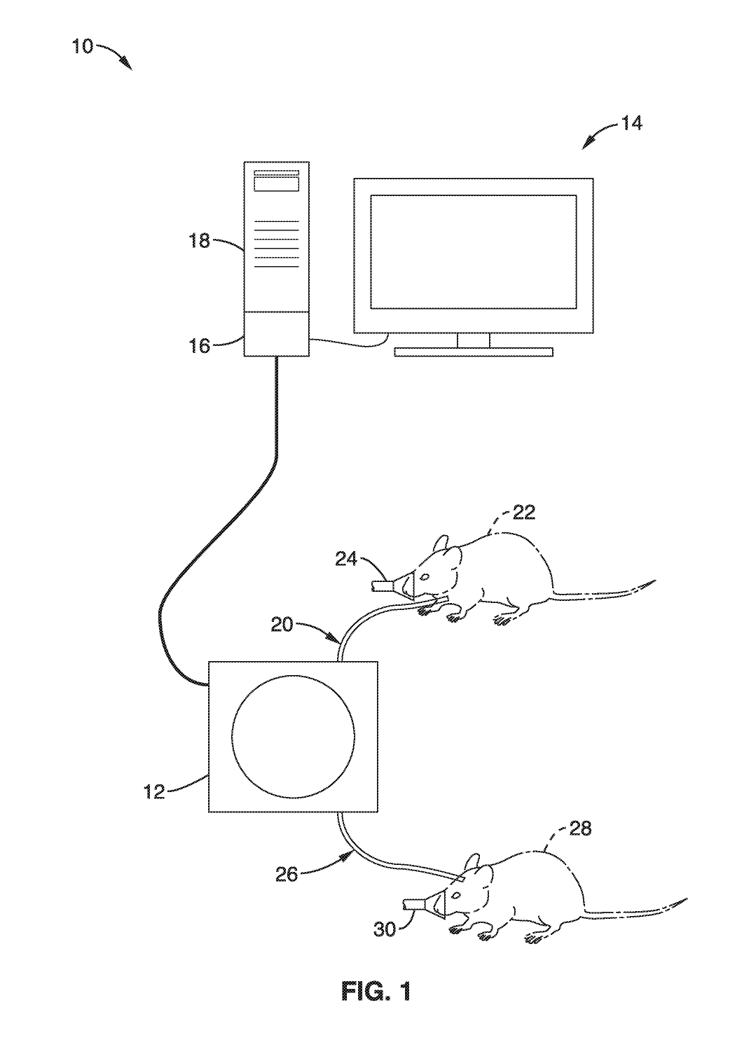

Referring more specifically to the drawings, for illustrative purposes, embodiments of an apparatus and computer controlled methods for heterochronic or isochronic blood or blood component exchange are generally shown. Embodiments of the technology are described generally in FIG. 1 through FIG. 3 to illustrate the characteristics and functionality of the apparatus and system. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, an apparatus and system for controlled small animal blood exchange is shown schematically. In contrast to the permanent anastomosis of parabiosis schemes, the blood exchange system 10 of FIG. 1 allows the animals to be connected and disconnected at will as well as removing the influence of shared organs, adaptation to being joined, etc. It can be seen that, compared with heterochronic parabiosis, heterochronic blood exchange in small animals is less invasive and enables better-controlled studies with more immediate translation to therapies for humans.

The controlled experimental system shown schematically in FIG. 1 has a microfluidic pump circuit 12 that is controlled by computer 14. The computer 14 has an optional display, data storage, a processor 16 and a non-transitory memory 18 storing programming instructions executable by the processor.

The programming of computer 14 can control blood volumes, flow rates, direction of transfer and frequency of transfer events. The computer 14 can also record time/date, subject animal identity and other experimental parameters and results as well as control anesthesia administration.

The pump circuit 12 preferably uses a peristaltic pump and input/output lines with a hollow needle to couple with the vasculature of the test subjects. In FIG. 1, the pump assembly 12 is connected with a first test subject 2 with a tubular transfer line 20 that can have central single or double channels in the line 20. Similarly, a tubular transfer line 26 is connected with a second test subject 28 and to the pump circuit 12. The transfer line preferably has a hollow needle to engage the circulatory system of the subject.

The first test subject is also sedated/oxygenated with a source of gases and mask 24 and the second test subject has a similar gas source and mask 30 to support the animals during experimental procedures.

A major design constraint of small animal blood manipulation that was addressed is the low volume of total blood that can be removed from a small animal at one time. It is not prudent to remove more than 10% of an animal's blood at once, and mice contain 5-8% (w/w) blood. This translates to approximately 150 μl of blood that can be removed from a 30 g mouse. Small volume microfluidic blood manipulation systems exist for lab on a chip and other diagnostic applications, however the apparatus is the first to allow for continuous blood flow as required for larger scale experimental applications in live mice, for example. In contrast to parabiosis, where joint circulation is established in approximately 7-10 days through the growth of skin capillaries, blood exchange is instantaneous and well controlled by the device. The procedure is also less invasive than parabiosis as it does not involve as much invasive surgery, only the catheterization of a jugular vein. The exchanged blood can be visualized in the tubing 20, 26 and the exchange volumes are easily measured.

The apparatus and methods described here enable blood exchange to be performed in mice and other small animals, thus allowing for well-controlled experimental tests, which can be rapidly translated to combating a number of age-related degenerative pathologies of muscle, brain, etc. using human-based exchange devices that are already FDA approved.

Small animal blood exchange and parabiosis are different in many ways, including the timing of blood exchange, the involvement of the immune system (i.e. parabiotic disease), the participation of the organ systems, and the environmental enrichment and pheromones, etc. However, many of the effects are quite similar to parabiosis and take place rapidly after the heterochronic blood exchange procedure, rather than being required to wait at least 7-10 days with parabiosis.

The apparatus and system 10 allows a wide variety of potential experiments and testing of potential treatments and possible therapeutic substances. The computer controlled microfluidic peristaltic pump circuit and computer controlled extracorporeal blood manipulation system can be used to discover the effects on young animals of a transfer of old blood as well as the effects on old animals of a transfer of young blood without surgically joining the animals. For example, the attenuation of liver fibrosis and adiposity, muscle wasting and neuro-degeneration of old animals can be observed and quantified. Likewise, many different experiments elucidating any deleterious effects of old blood on a young subject can also be performed using the blood exchange system.

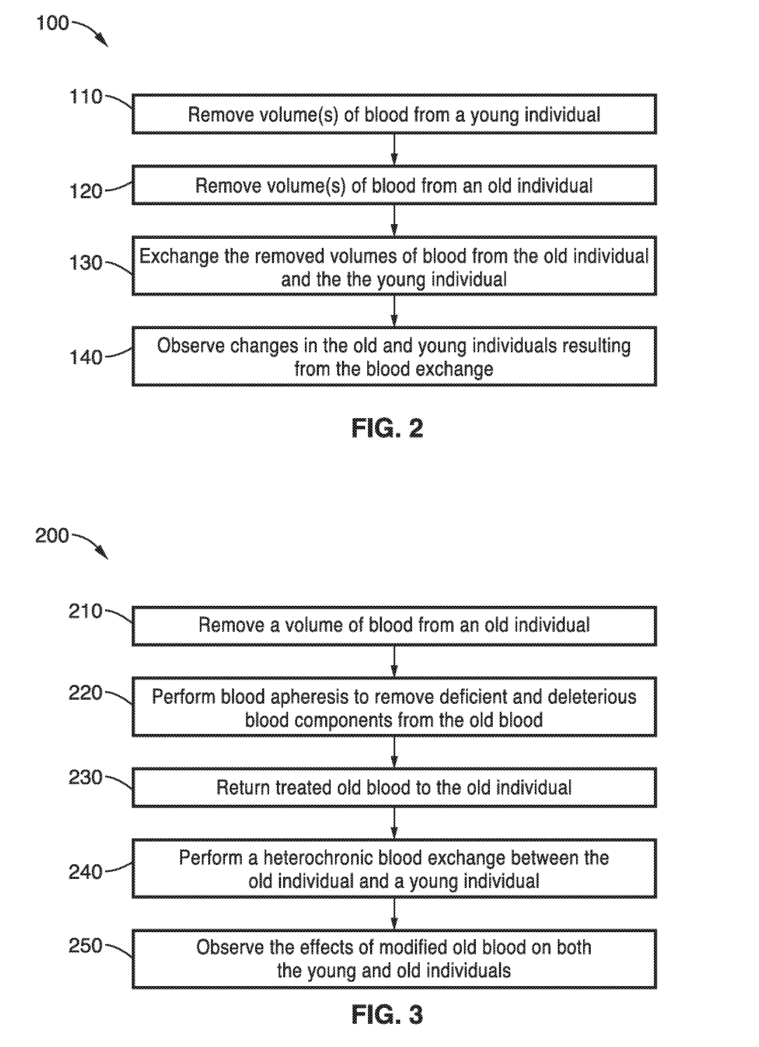

FIG. 2 is a flow diagram of one embodiment of a method 100 for using the apparatus to evaluate the effect of heterochronic blood exchange on old and young subjects as well as blood exchanges between individuals of the same age for control purposes. Young individuals are defined as animals that are within the first third of their life expectancy and old individuals are defined as being within the final third of their life expectancy.

In this illustration, a total volume of young blood of a young subject is removed in one or more increments at block 110 of FIG. 2. A total volume of old blood from an old subject is also removed in one or more increments at block 120. The removed volumes of blood from the subjects at block 110 and 120 are exchanged at block 130. The removal and exchange events from the old and young subjects can preferably alternate so that there are no large fluctuations in the blood volumes of either subject. In the preferred embodiment, the exchange of blood between individuals is virtually constant. The total volume of blood transferred and the overall time of transfer can vary and can be selected to optimize any physiological changes that are observed.

Finally, at block 140, the old and young test subjects are evaluated to observe any changes that have occurred as a result of the blood exchange. The observed can also be compared with the results of isochronic blood exchanges between two individuals of approximately the same age.

Another illustration of the types of experiments that can be carried out with the apparatus is shown schematically in FIG. 3. In this illustration, the old blood from the old subject is modified with the removal of negative factors and/or the addition of positive factors prior to heterochronic blood exchange.

At block 210 of FIG. 3, a volume of old blood is removed from the old individual. The volume blood removed is preferably 10% or less of the total blood of the animal in any transfer event. Blood apheresis of the old blood is performed at block 220 to remove specific deficient or deleterious blood components from the old blood. The steps at block 210 and 220 can be repeated so that more than 10% of the old blood has been treated. The treated old blood is returned to the old subject at block 230.

Heterochronic blood exchange is then performed between old and young subjects with the apparatus at block 240. Any effects of the modified old blood on the old and young subjects are then observed at block 250. The results can optionally be compared with results of a control isochronic blood exchange between individuals of approximately the same age with one having modified blood and the other with no modifications.

In other embodiments, the modification to the old blood may be the addition of a suspected positive factor with or without the removal of a negative factor to see any effect resulting from the presence of the suspected positive factor.

It can be seen that many different experimental methods can be contrived for use with the apparatus and system. For example, the apparatus can also be used with procedures that allow the identification of the blood factor(s) that rapidly influence the health of several tissues and uncover their mechanism of action. In addition, embodiments of the apparatus that are configured to separate certain blood components will allow experiments based on heterochronic plasma exchanges such as separate the effects of plasma from those of leukocytes. Thus, blood exchange in small animals rather than parabiosis enables well-controlled studies with more rapid translation for the development of therapies for humans.

The technology described herein may be better understood with reference to the accompanying examples, which are intended for purposes of illustration only and should not be construed as in any sense limiting the scope of the technology described herein as defined in the claims appended hereto.

### EXAMPLE 1

In order to demonstrate the operational principles of the apparatus and methods of use, a computer controlled microfluidic peristaltic pump circuit and computer controlled extracorporeal blood manipulation system as generally depicted in FIG. 1 was constructed. Using the small animal apparatus, blood was exchanged between 4 pairs of young to old mice, using 4 pairs of isochronic, young to young exchanges, and 4 pairs of isochronic, old to old exchanges, as controls. Virtually 100% animal viability was maintained when two series of 15 exchanges of 150 μl of blood per series were performed over the course of 24 hours, establishing a blood equilibrium similar to parabiosis between the pairs in a fraction of the time. This regiment was employed for the studies and will be referred to as a single procedure of blood exchange hereafter.

First a jugular venous catheter was inserted in the right jugular vein of each animal. Using a 10 μl Hamilton syringe 10 μl of catheter locking solution containing 500 units per ml lithium heparin in 90% glycerol and 10% phosphate buffered saline (PBS) were introduced into the catheter to prevent clot formation. The animals were allowed to heal from the surgical procedure for 24 hours and then they were immobilized with isoflurane anesthesia at a 1% concentration. The locking solution was removed from their catheters and a bolus of PBS containing 0.5 units per μl lithium heparin was administered IV at 100 units per kg and they were connected to our blood exchange apparatus. Briefly, using the microfluidic blood exchange device 150 μl of blood was transferred from one mouse to another 15 times with a 30 second delay between blood administration and withdrawal twice within 24 hours to yield a greater than 90% homogenization of blood between the 2 animals. The blood coming from one animal and going into another was visualized and it was calculated that after a single exchange session the blood of the two animals is approximately 90% homogenized, and are able to program precisely the degree of homogenization we desire and confirm the volumes of exchanged blood in each experiment (knowing the tubing diameter and length). The mice were weighed before the exchanges and the volumes of exchanged blood was calculated based on fluid dynamics as well as the outcome of many control experiments in which total blood volume is correlated with animal weight.

### EXAMPLE 2

With the test bed established, experiments generally following the sequence of FIG. 2 could be conducted to demonstrate the variety of experiments or tests that can be performed with the blood exchange system.

To examine the effect of young blood on old muscle regeneration, the apparatus was used to exchange blood between young and old test subjects. The efficiency of muscle regeneration was determined in a manner identical to heterochronic parabiosis studies for comparison.

One day after the blood exchange, Tibiallis Anterior (TA) hind leg muscles of all mice were injured by cardiotoxin (CTX) and 5 days later this muscle, as well as non-injured livers and brains were isolated postmortem. TA muscles were injected with cardiotoxin 1 (CTX, Sigma, 0.1 mg/ml). 10 micron muscle cryo-sections were prepared from TA muscle, which was isolated at 5 days post CTX injury. These cryo-sections were analyzed by Hematoxylin and Eosin (H&E), staining and by eMyHC immuno-detection followed by microscopy and quantification of the percent of de novo small eMyHC+ myofibers with centrally-located nuclei that robustly appear in young, but are less in the old injured muscle, which typically shows more inflammation and incipient fibrosis.

It was observed that a single procedure of heterochronic blood exchange significantly improved the regeneration of old muscle after experimental injury (both when assayed by H&E staining or eMyHC immunofluorescence), while there was no statistically significant decline in the robust regeneration of the young muscle. The numbers of de-novo myofibers were slightly higher for all cohorts when counted by the more robust eMyHC immunofluorescence method as compared to H&E, but the relative differences between the YY, YO, OY and OO cohorts remained virtually the same.

The de-novo fiber size was also evaluated. As expected, the regenerating fiber size declined with age but fiber size was increased in old mice transfused with young blood, while remaining unchanged in young mice transfused with old blood. Importantly, the degree of fibrosis (a culprit of muscle aging) was also reduced by the young blood exchange and was not increased in the young mice transfused with old blood.

### EXAMPLE 3

To further demonstrate the testing methods that are available with the apparatus, the effect of old blood on the functional performance of young test subjects was illustrated. To assay functional performance, the blood exchange studies were repeated without muscle injury, and a four-limb hanging test was applied to the isochronic and heterochronic cohorts 6 days after the blood exchange (e.g. the same time frame when muscle repair was studied in the injured mice). Animal strength, endurance and learning are all measured in this test. In particular, mice hang inverted from a 1 cm mesh screen over soft bedding, and the time until the mouse drops is recorded over three trials, and the maximal time multiplied by the weight is expressed as hanging index. Interestingly, exchange with old blood markedly diminished the maximal hanging index of young animals (3 out of 4 mice) but there was no increase in this parameter for the old mice transfused with young blood. Of note, the initial hanging indices in training session were not significantly different between the young and old mice, but young animals transfused with young blood became statistically better than the old mice after the training session, while young mice transfused with old blood remained statistically undistinguishable from the old cohorts.

These data extrapolate the findings obtained with heterochronic parabiosis, and establish that the beneficial effects of young blood for the regeneration of old muscle take place right away and without the contribution of young organ systems or altered activity levels between the isochronic and heterochronic pairs. Moreover, while one exchange of young blood improves muscle regeneration in old animals, it does not improve the functional performance as measured by the hanging test, while in young animals the functional performance declines very rapidly after one exchange of old blood.

### EXAMPLE 4

Another demonstration of the system was with an evaluation of efficiency of hippocampal neurogenesis in response to old blood and muscle injury. For this evaluation, mouse brains were collected and sectioned at 25 micron using a cryostat. Sections were fixed in 4% paraformaldehyde and immunostained with antibodies to Ki67, using Hoechst co-stain to detect all nuclei. The numbers of Ki67+ proliferating SGZ cells were quantified throughout the entire Dentate Gyrus of the hippocampus. It was observed that the Ki67+ SGZ cells were virtually all-neural stem cells based on the co-immunodetection of Sox-2 results.

Based on cell numbers of either SGZ Ki67+ or SGZ Ki67+/Sox2+, it was shown that one exchange of heterochronic blood severely decreased hippocampal neurogenesis in young mice, and surprisingly, there was no significant positive effect in the old mice that had been exchanged with young blood.

The influences of heterochronic blood exchange on neurogenesis in the presence and absence of muscle injury were compared to assay for potential changes in the brain that might be caused by additional stress and peripheral inflammation. While a statistically significant inhibition of young neurogenesis by old blood persisted, its magnitude was less in the absence of muscle injury. There was no enhancement of old neurogenesis by the young blood, with or without muscle injury. These data confirm the negative effects of the old systemic milieu on neurogenesis in young hippocampi and demonstrate that such inhibition is very rapid and is uncoupled from influences of old organ systems, pheromones and changes in the environmental stimulation or exercise. Muscle injury after blood exchange might add to the magnitude of the negative effects of old blood on young neurogenesis, and even without muscle injury, young hippocampal neurogenesis quickly declines after one old blood exchange.

### EXAMPLE 5

A further demonstration of the system and potential methods was with an assay of the efficiency of ongoing hepatogenesis in mice that were and were not experimentally injured in a muscle by the co-immunodetection of a proliferation marker, Ki67, and a hepatocyte marker albumin, in 10 micron liver cryosections. The liver was shown to respond to heterochronic blood exchange and muscle injury.

It was observed that the numbers of proliferating old hepatocytes were increased after a single procedure of heterochronic blood exchange, while the numbers of Ki67+albumin+ young hepatocytes declined upon transfusion of old blood. The ongoing hepatogenesis in animals that did not experience muscle injury was much less prominent even in young mice, suggesting that hepatogenesis increases during muscle repair, but still the heterochronic effects of a single blood exchange were observed.

Many fibrotic areas were seen in old livers, which at times had proliferating clusters of small albumin negative cells. Such areas were not present in young livers, and very interestingly the numbers of these fibrotic proliferative clusters declined in the livers of old animals that were exchanged with young blood, regardless of whether muscle was or was not injured.

As another metric for improvement in liver health, liver tissue adiposity by Oil Red staining on 10 micron cryosections from the above-described animals was assayed. Old livers were markedly more positive for Oil Red, as compared to young and interestingly, transfusion with young blood somewhat reduced old liver adiposity, while there was no significant increase in young liver adiposity. These results demonstrate that heterochronic blood exchange and heterochronic parabiosis yield similar enhancement of old hepatogenesis and decline of young hepatogenesis; and suggest that muscle damage enhances ongoing hepatogenesis in young mice. Additionally, the fibrotic regions and adiposity rapidly decline in old livers after the exposure to young blood. Such effects manifest after just a single procedure of blood exchange and in the absence of the influences from heterochronic organ systems.

### EXAMPLE 6

To illustrate another type of use of the apparatus, experiments directed to identifying the molecular mechanisms that are responsible for these rapid influences of circulation on tissue repair and maintenance by looking at levels of B2M. B2M is the invariant chain of MHC class I that becomes elevated with inflammation and is believed to be over-pronounced in old muscle and brain, as compared to young.

B2M levels were assayed by immunofluorescence in tissue cryosections and by Western Blotting in the young and old mice that underwent isochronic versus heterochronic blood exchange. For tissues derived from mice injured with CTX in their TAs, the immunofluorescence on muscle and brain tissue cryosections demonstrated that exchange with old blood rapidly (within 6 days), elevated the B2M levels in young muscle located outside of the CTX injury, and in the SGZ of the young hippocampus. Interestingly, the B2M remained high in the old hippocampi of the heterochronically exchanged animals.

Furthermore, these age-specific differences in B2M in muscle were less pronounced between YY and YO cohorts and were undetectable between the OO and OY cohorts when immunofluorescence was performed at the sites of CTX injury. Muscle regeneration was in agreement with the previous findings that inflammation overlaps in space and time with muscle repair and that some degree of transient inflammation is needed for successful myogenesis.

Western Blotting confirmed the results obtained by the immunofluorescence and demonstrated that B2M levels were increased with age in muscle and in brains, while there was no detectable age-specific increase of B2M in livers. The regional tissue differences in B2M levels are not resolvable by the Western analyses, thus the differences between the cohorts were less drastic, but in general agreement with those seen by the immunofluorescence.

The age-elevated increase of B2M was less noticeable in the muscle and brains of the animals that did not experience experimental muscle injury; for livers there was again no detectable age-specific change. By immunofluorescence, the regional (DG) age-specific difference in B2M persisted in brains of young versus old mice that did not experience muscle injury; and no significant modulation of B2M were detected between YY versus YO or OO versus OY cohorts.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that that the present disclosure encompasses multiple embodiments which include, but are not limited to, the following:

1. An apparatus for small animal blood exchange research, comprising: (a) a first blood transfer line with coupling configured to couple with vasculature of a first subject; (b) a second blood transfer line with coupling configured to couple with vasculature of a second subject; (c) a pump assembly operably connected with the first and second blood transfer lines; (d) a processor controlling the pump assembly; and (e) a non-transitory memory storing instructions executable by the processor; (f) wherein the instructions, when executed by the processor, perform steps comprising: (i) removing at least one volume of blood from the first subject; (ii) removing at least one volume of blood from the second subject; (iii) replacing the removed volume of blood from the first subject with the volume of blood from the second subject; and (iv) replacing the removed volume of blood from the second subject with the volume of blood from the first subject.

2. The apparatus of any preceding or following embodiment, wherein the instructions when executed by the processor further perform steps comprising: controlling a rate of removal of each volume of blood removed from the first subject; controlling a rate of replacement of each volume of blood replaced in the first subject; controlling a rate of removal of each volume of blood removed from the second subject; and controlling a rate of replacement of each volume of blood replaced in the second subject.

3. The apparatus of any preceding or following embodiment, wherein the instructions when executed by the processor further perform steps comprising: controlling a number of removal and replacement events of blood from the first subject over a time; and controlling a number of removal and replacement events of blood from the second subject over time.

4. The apparatus of any preceding or following embodiment, wherein the instructions when executed by the processor further perform steps comprising: controlling a time between removal and replacement events of blood from the first subject; and controlling a time between removal and replacement events of blood from the second subject over time.

5. The apparatus of any preceding or following embodiment, wherein the instructions when executed by the processor further perform steps comprising: controlling a total volume of blood removed from the first subject; controlling a total volume of blood replaced in the first subject; controlling a total volume of blood removed from the second subject; and controlling a total volume of blood replaced in the second subject.

6. The apparatus of any preceding or following embodiment, wherein the instructions when executed by the processor further perform steps comprising: recording removal and replacement events of blood from the first subject; and recording removal and replacement events of blood from the second subject.

7. The apparatus of any preceding or following embodiment, wherein the first blood transfer line and the second transfer line comprise; a central input/output channel; and a hollow needle coupled to the input/output channel of the transfer line; wherein the pump assembly removes blood and replaces blood through the input/output channel.

8. The apparatus of any preceding or following embodiment, wherein the first blood transfer line and the second transfer line comprise; an input channel; an output channel; a hollow needle coupled to the input and output channels of the transfer line; wherein the pump assembly removes blood through the input channel and replaces blood through the output channel.

9. The apparatus of any preceding or following embodiment, wherein the pump assembly comprises a peristaltic pump.

10. A method for investigating animal aging and age related diseases, the method comprising: (a) performing heterochronic blood exchange between an old subject and a young subject with an apparatus comprising: (i) a first blood transfer line with coupling configured to couple with vasculature of a young subject; (ii) a second blood transfer line with coupling configured to couple with vasculature of an old subject; (iii) a pump assembly operably connected with the first and second blood transfer lines; (iv) a processor controlling the pump assembly; and (v) a non-transitory memory storing instructions executable by the processor; and (b) analyzing physiology of the old subject after heterochronic blood exchange.

11. The method of any preceding or following embodiment, further comprising: analyzing physiology of the young subject after heterochronic blood exchange.

12. The method of any preceding or following embodiment, further comprising: performing isochronic blood exchange between a first subject and a second subject; comparing physiology of the first or second subjects with the physiology of the old subject after heterochronic blood exchange; and comparing physiology of the first or second subjects with the physiology of the young subject after heterochronic blood exchange.

13. The method of any preceding or following embodiment, further comprising: repeating the heterochronic blood exchange more than one time prior to analyzing physiology of the old subject and the young subject.

14. The method of any preceding or following embodiment, further comprising: repeating the isochronic blood exchange more than one time prior to analyzing physiology of the first subject or second subject; and repeating the heterochronic blood exchange more than one time prior to analyzing physiology of the old subject and the young subject.

15. A method for investigating treatment and prevention of age-related diseases, the method comprising: (a) removing one or more volumes of blood from an old individual; (b) performing blood apheresis to remove deficient and deleterious blood components from the volumes of old blood;(c) returning treated old blood to the old individual; and (d) performing heterochronic blood exchange between the old subject and a young subject with an apparatus comprising:(i) a first blood transfer line with coupling configured to couple with vasculature of a young subject;(ii) a second blood transfer line with coupling configured to couple with vasculature of the old subject;(iii) a pump assembly operably connected with the first and second blood transfer lines; (iv) a processor controlling the pump assembly; and (v) a non-transitory memory storing instructions executable by the processor; and (e) analyzing physiology of the old subject after heterochronic blood exchange.

16. The method of any preceding or following embodiment, further comprising: performing isochronic blood exchange between a first subject and a second subject; comparing physiology of the first or second subjects with the physiology of the old subject after heterochronic blood exchange; and comparing physiology of the first or second subjects with the physiology of the young subject after heterochronic blood exchange.

17. The method of any preceding or following embodiment, wherein the instructions when executed by the processor further perform steps comprising: controlling a rate of removal of each volume of blood removed from the first subject; controlling a rate of replacement of each volume of blood replaced in the first subject; controlling a rate of removal of each volume of blood removed from the second subject; and controlling a rate of replacement of each volume of blood replaced in the second subject.

18. The method of any preceding or following embodiment, wherein the instructions when executed by the processor further perform steps comprising: controlling a number of removal and replacement events of blood from the first subject over a time; and controlling a number of removal and replacement events of blood from the second subject over time.

19. The method of any preceding or following embodiment, wherein the instructions when executed by the processor further perform steps comprising: controlling a time between removal and replacement events of blood from the first subject; and controlling a time between removal and replacement events of blood from the second subject over time.

20. The method of any preceding or following embodiment, wherein the instructions when executed by the processor further perform steps comprising: controlling a total volume of blood removed from the first subject; controlling a total volume of blood replaced in the first subject; controlling a total volume of blood removed from the second subject; and controlling a total volume of blood replaced in the second subject.

21. A method for attenuation of liver fibrosis and adiposity, muscle wasting and neuro-degeneration, the method comprising: (a) removing a volume of blood from a young individual; (b) removing a volume of blood from an old individual; and (c) replacing the removed volume of blood from the old individual with the volume of blood from the young individual; (d) wherein rapid liver regeneration, reduction of fibrosis, and muscle healing post-injury or immobility, or old age are induced.

22. A method for treatment and prevention of age-related diseases, the method comprising: (a) removing a volume of blood from an old individual; (b) performing blood apheresis to remove deficient and deleterious blood components from the volume of blood; (c) returning treated blood to the old individual; and (d) performing a heterochronic blood exchange between the old individual and a young individual.

23. The method of any preceding or following embodiment, further comprising: repeating the heterochronic blood exchange at regular intervals over time.

24. The method of any preceding or following embodiment, further comprising: repeating the blood apheresis at regular intervals over time.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for small animal blood exchange research, comprising:
(a) a first blood transfer line with coupling configured to couple with vasculature of a first subject;
(b) a second blood transfer line with coupling configured to couple with vasculature of a second subject;
(c) a pump assembly operably connected with the first and second blood transfer lines;
(d) a processor controlling the pump assembly; and
(e) a non-transitory memory storing instructions executable by the processor;
(f) wherein said instructions, when executed by the processor, perform steps comprising:
(i) removing at least one volume of blood from the first subject;
(ii) removing at least one volume of blood from the second subject;
(iii) replacing the removed volume of blood from the first subject with the volume of blood from the second subject; and
(iv) replacing the removed volume of blood from the second subject with the volume of blood from the first subject.

2. The apparatus of claim 1, wherein said instructions when executed by the processor further perform steps comprising:
controlling a rate of removal of each volume of blood removed from the first subject;
controlling a rate of replacement of each volume of blood replaced in the first subject;
controlling a rate of removal of each volume of blood removed from the second subject; and
controlling a rate of replacement of each volume of blood replaced in the second subject.

3. The apparatus of claim 1, wherein said instructions when executed by the processor further perform steps comprising:
controlling a number of removal and replacement events of blood from the first subject over a time; and
controlling a number of removal and replacement events of blood from the second subject over time.

4. The apparatus of claim 3, wherein said instructions when executed by the processor further perform steps comprising:
controlling a time between removal and replacement events of blood from the first subject; and
controlling a time between removal and replacement events of blood from the second subject over time.

5. The apparatus of claim 1, wherein said instructions when executed by the processor further perform steps comprising:
controlling a total volume of blood removed from the first subject;
controlling a total volume of blood replaced in the first subject;
controlling a total volume of blood removed from the second subject; and
controlling a total volume of blood replaced in the second subject.

6. The apparatus of claim 1, wherein said instructions when executed by the processor further perform steps comprising:
recording removal and replacement events of blood from the first subject; and
recording removal and replacement events of blood from the second subject.

7. The apparatus of claim 1, wherein said first blood transfer line and said second transfer line comprise;
a central input/output channel; and
a hollow needle coupled to the input/output channel of the transfer line;
wherein said pump assembly removes blood and replaces blood through the input/output channel.

8. The apparatus of claim 1, wherein said first blood transfer line and said second transfer line comprise;
an input channel;
an output channel;
a hollow needle coupled to the input and output channels of the transfer line;
wherein said pump assembly removes blood through the input channel and replaces blood through the output channel.

9. The apparatus of claim 1, wherein said pump assembly comprises a peristaltic pump.

10. A method for investigating animal aging and age related diseases, the method comprising:
(a) performing heterochronic blood exchange between an old subject and a young subject with an apparatus comprising:
(i) a first blood transfer line with coupling configured to couple with vasculature of a young subject;
(ii) a second blood transfer line with coupling configured to couple with vasculature of an old subject;
(iii) a pump assembly operably connected with the first and second blood transfer lines;
(iv) a processor controlling the pump assembly; and
(v) a non-transitory memory storing instructions executable by the processor; and
(b) analyzing physiology of the old subject after heterochronic blood exchange.

11. The method of claim 10, further comprising:
analyzing physiology of the young subject after heterochronic blood exchange.

12. The method of claim 10, further comprising:
performing isochronic blood exchange between a first subject and a second subject;
comparing physiology of the first or second subjects with the physiology of the old subject after heterochronic blood exchange; and
comparing physiology of the first or second subjects with the physiology of the young subject after heterochronic blood exchange.

13. The method of claim 11, further comprising:
repeating the heterochronic blood exchange more than one time prior to analyzing physiology of the old subject and the young subject.

14. The method of claim 12, further comprising:
repeating the isochronic blood exchange more than one time prior to analyzing physiology of the first subject or second subject; and
repeating the heterochronic blood exchange more than one time prior to analyzing physiology of the old subject and the young subject.

15. A method for investigating treatment and prevention of age-related diseases, the method comprising:
(a) removing one or more volumes of blood from an old individual;
(b) performing blood apheresis to remove deficient and deleterious blood components from the volumes of old blood;
(c) returning treated old blood to the old individual; and
(d) performing heterochronic blood exchange between the old subject and a young subject with an apparatus comprising:
(i) a first blood transfer line with coupling configured to couple with vasculature of a young subject;
(ii) a second blood transfer line with coupling configured to couple with vasculature of the old subject;
(iii) a pump assembly operably connected with the first and second blood transfer lines;
(iv) a processor controlling the pump assembly; and
(v) a non-transitory memory storing instructions executable by the processor; and
(e) analyzing physiology of the old subject after heterochronic blood exchange.

16. The method of claim 15, further comprising:
performing isochronic blood exchange between a first subject and a second subject;
comparing physiology of the first or second subjects with the physiology of the old subject after heterochronic blood exchange; and
comparing physiology of the first or second subjects with the physiology of the young subject after heterochronic blood exchange.

17. The method of claim 16, wherein said instructions when executed by the processor further perform steps comprising:
controlling a rate of removal of each volume of blood removed from the first subject;
controlling a rate of replacement of each volume of blood replaced in the first subject;
controlling a rate of removal of each volume of blood removed from the second subject; and
controlling a rate of replacement of each volume of blood replaced in the second subject.

18. The method of claim 16, wherein said instructions when executed by the processor further perform steps comprising:
controlling a number of removal and replacement events of blood from the first subject over a time; and
controlling a number of removal and replacement events of blood from the second subject over time.

19. The method of claim 18, wherein said instructions when executed by the processor further perform steps comprising:
controlling a time between removal and replacement events of blood from the first subject; and
controlling a time between removal and replacement events of blood from the second subject over time.

20. The method of claim 16, wherein said instructions when executed by the processor further perform steps comprising:
controlling a total volume of blood removed from the first subject;
controlling a total volume of blood replaced in the first subject;
controlling a total volume of blood removed from the second subject; and
controlling a total volume of blood replaced in the second subject.
